(19) United States
(12) Patent Application Publication
(10) Pub. No.: US 2019/0217048 A1
(43) Pub. Date: Jul. 18, 2019

(54) SLEEP-AID DEVICE AND METHOD THEREOF

(71) Applicant: DreamJay, Inc., Warsaw (PL)

(72) Inventors: Lukasz Mlodyszewski, Warsaw (PL); Kornel Rostek, Warsaw (PL)

(21) Appl. No.: 16/131,304

(22) Filed: Sep. 14, 2018

Related U.S. Application Data
(60) Provisional application No. 62/558,808, filed on Sep. 14, 2017.

(51) Int. Cl. A61M 21/02; A61B 5/00

(52) U.S. Cl. CPC A61M 21/02; A61M 2021/0027; A61B 5/4815; A61B 5/4812

(57) ABSTRACT

A sleep-aid device may include at least one sensor configured to sense an activity of a user during sleep, a memory unit, and a processing unit in communication with the at least one sensor and the memory unit. Audiovisual and audio content may be available to be selected for display on an electronic display and selected for play by an audio output device. The processing unit may be configured to select a common theme of audiovisual content and audio content, enable the user to play the audiovisual content on the electronic display, initiate playing the first audio content by the audio output device a determined number of times to ease falling asleep, cause the second audio content to be played by the audio output device, and cause the third audio content to be played at a first volume threshold to wake the user by the audio output device.

### RELATED APPLICATIONS

This application claims priority to co-pending U.S. Provisional Patent Application having Ser. No. 62/558,808 filed on Sep. 14, 2017; the contents of which are incorporated herein in their entirety.

### BACKGROUND

Sleep disorders are problematic in today's fast-pace society. People spend almost one-third of their lives sleeping so sleep is important for both health and quality of life. Furthermore, people often suffer from various disorders relating to sleep. Much of the population (estimated to be 30%-40%) has problems falling asleep, and many (estimated at 5%-8%) suffer from frequent nightmares (three times per week and more). Unfortunately, as few as one nightmare per week may cause someone significant distress or impairment in important areas of functioning. Nightmares are associated with disturbed sleep, low well-being, and affect daytime mood and behavior. In some cases, nightmares can be a contributing factor to alcohol and substance abuse, suicidal ideation, or completed suicide. As such, nightmares can have a profound negative effect on an individual's mental and physical health and quality of life.

Nightmare disorder is characterized by repeated awakenings from sleep with detailed recall of extended and extremely frightening dreams, usually involving threats to survival, security, or self-esteem. A particularly hard hit cross-section of people that have frequent nightmares are military members and veterans. Large numbers of post-traumatic stress disorder patients experience nightmares. Some medications may induce frightening dreams, either during treatment or following withdrawal. Even though sleeping issues are serious and massive problems, it is difficult to find accessible and effective solutions. As a result, the global market for sleep aids is on track to be a multi-billion dollar industry. Unfortunately, many of these solutions are impractical, ineffective, and/or detrimental.

Sleep disorder studies typically involve patients that may have a wide range of symptoms and results on the patients. The studies often measure body functions, including brain activity (electroencephalography (EEG)), eye movement, snoring, and others. Additional signals are often included, depending on polysomnography (PSG) purpose, including heart rhythm (electrocardiogram (ECG)). While sleep disorder studies have been helpful for researchers, the cost for a highly skilled and trained researcher who can identify sleep disorder patterns and provide effective treatment is prohibitive. Moreover, for the large percentage of the population with sleep disorders, the ability to use such specialized equipment is impracticable, especially given that a single night of sleep evaluation with such equipment may cost $10,000 or more. However, even with such equipment, the ability for even trained sleep disorder professionals to analyze sleeping patterns based on measurements therefrom can be challenging.

Attempts to address sleep disturbance issues include lifestyle and therapeutic strategies, such as encouraging the practice of good sleep habits, cognitive behavioral therapy, adjunctive therapy, image rehearsal therapy (IRT), hypnosis, drug therapy, and pharmacologic and behavioral interventions. Additionally, sleep aid devices, such as actigraphs, headgear, watches, and other sensor based clinical equipment have been used in an effort to help sufferers of sleep disorders. However, existing technology fails (i) to continually adapt solutions to specific users, and (ii) to suitably address an entire sleep cycle, thereby failing to provide meaningful improvements to an individual with a sleep disorder. As such, a comprehensive and science-based approach is still needed to help sufferers with sleep disorders.

### SUMMARY OF THE INVENTION

A sleep-aid device may be configured to provide audiovisual and audio content to a user to aid falling asleep, staying asleep, preventing nightmares, and waking up. The audiovisual and audio content may share a common theme that may optimally assist in falling asleep and maintaining a quality sleep. The sleep-aid device may be configured to predict a state of sleep based on signals representative of biological activity during sleep. The sleep-aid device may play audio content based on the predicted state of sleep. The sleep-aid device may include biological activity sensors configured to sense biological activity and communicate a signal representative of the biological activity. The sleep-aid device may control and continually optimize volume, length, frequency, and other playback parameters in response to increasing data regarding a single sleep session and/or a group of sleep sessions. Statistical analysis and signal processing may be applied to the signals representative of biological activity to utilize the data in modifying playback so that the sleep-aid device is most efficient at assisting each individual user during sleep.

One embodiment of a sleep-aid device may include at least one sensor configured to sense an activity of a user during sleep, a memory unit configured to store audiovisual and audio content, and a processing unit in communication with the sensor(s) and the memory unit. The audiovisual and audio content may be available to be (i) selected for display on an electronic display and (ii) selected for play by an audio output device. The processing unit may be configured to select a common theme of audiovisual content and audio content and enable the user to play the audiovisual content on the electronic display. After the audiovisual content has completed, the processing unit may initiate playing the first audio content by the audio output device a determined number of times to ease falling asleep. In response to receiving a first sensor signal from one of the sensor(s) that senses a first biological activity indicative of a specific state during sleep, the processing unit may cause the second audio content to be played by the audio output device. In response to receiving a second sensor signal from one of the sensor(s) that senses a second biological activity indicative of the user beginning to wake up, the processing unit may cause the third audio content to be played at a first volume threshold to wake the user by the audio output device. The common theme of audiovisual and audio content may include a first audio content, a second audio content, and a third audio content.

One embodiment of a computer-implemented method of aiding sleep may include selecting a common theme of audiovisual content and audio content and enabling a user to play the audiovisual content. After the audiovisual content has completed, the method may include initiating playing the first audio content by an audio output device a determined number of times to ease falling asleep. In response to receiving a first sensor signal from one of at least one sensor that senses a first biological activity indicative of a specific state during sleep, the method may include causing the second audio content to be played by the audio output device. In response to receiving a second sensor signal from one of the sensor(s) that senses a second biological activity indicative of the user beginning to wake up, the method may include causing the third audio content to be played at a first volume threshold to wake the user by the audio output device. The common theme of audiovisual and audio content may include a first audio content, a second audio content, and a third audio content.

### BRIEF DESCRIPTION OF THE DRAWINGS

Illustrative embodiments of the present invention are described in detail below with reference to the attached drawing figures, which are incorporated by reference herein and wherein:

FIG. 1 is an illustration of an illustrative a sleep-aid environment inclusive of a sleep-aid device to aid a user while sleeping on a moveable surface, such as a mattress that moves in response to movement of the user;

FIG. 2 is a flow diagram of an illustrative process of aiding sleep based on acquisition of biological activity of a user through sensors;

FIG. 3A and 3B are flow diagrams of an illustrative process that represents a general flow of a sleep-aid system;

FIG. 4 is a flow diagram of an illustrative process of determining biological activity based on detecting motion of a sleeper;

FIG. 5 is a flow diagram of an illustrative process of determining a state of sleep based on statistical analysis of biological activity;

FIG. 6 is a flow diagram of an illustrative process of determining a playback counter value based on a predicted state of sleep;

FIG. 7 is a flow diagram of an illustrative a process of determining a volume to play an audio content in response to biological response of a user to the audio content;

FIG. 8 is an illustration of electronics of a sleep-aid system configured to aid a user while sleeping;

FIG. 9 is a block diagram inclusive of illustrative software modules that may be executed by a processing unit of a sleep-aid system according to FIG. 8 that is configured to aid a user in sleeping;

FIG. 10 is an illustration of illustrative screenshots of an application on a sleep-aid device configured to interface with a user regarding a sleep-aid application of the sleep-aid device; and

FIG. 11 is an illustration of an illustrative graph representative of a sleep session of a user inclusive of detected movement, predicted sleep states and/or activity based on the detected movement, and sound stimulations.

### DETAILED DESCRIPTION OF THE INVENTION

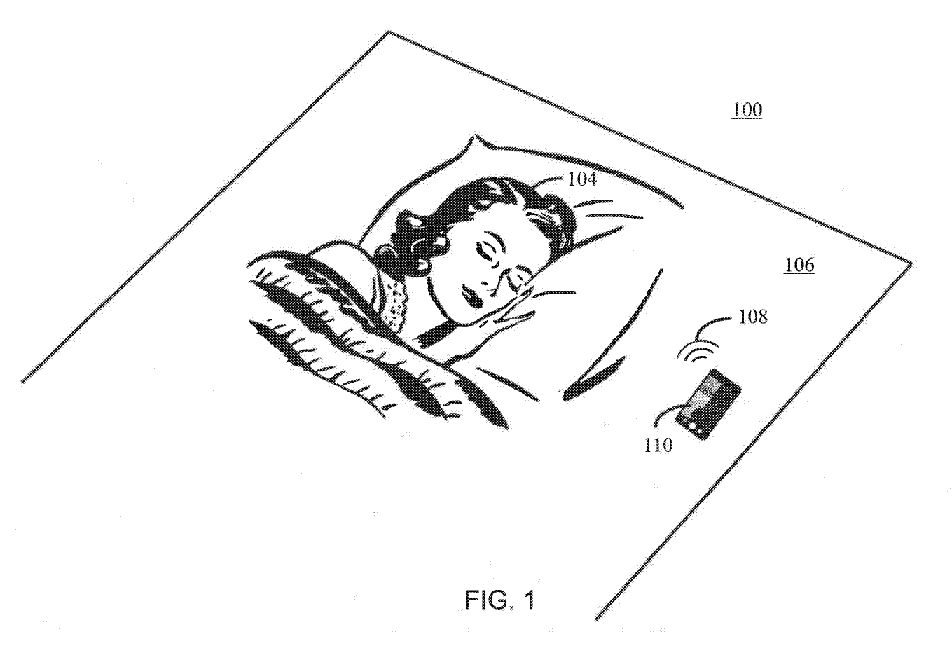

With regard to FIG. 1, an illustration of an illustrative a sleep-aid environment 100 inclusive of a sleep-aid device 102 to aid a user 104 while sleeping on a moveable surface 106, such as a mattress that moves in response to movement of the user 104, is shown to be placed on the moveable surface 106. In an embodiment, the sleep-aid device 102 may be an electronic device (e.g., smartphone, gaming device, dedicated sleep-aid device, etc.) configured to perform certain specialized functions, as further described herein, that assist the user 104 with a sleep disorder with sleeping, as further described herein. The sleep-aid device 102 may be configured to play audio content 108 and audiovisual content 110 to aid the user 104 in at least one of falling asleep, staying asleep, preventing nightmares, and waking up. In one embodiment, the sleep-aid device 102 may play the audiovisual content 110 followed by at least one audio content 108. In one embodiment, the at least one audio content 108 may include a first audio content, a second audio content, and a third audio content. As has been learned through extensive investigation, users with sleep disorders respond well to common themes of audiovisual and audio content. Different themes may be available for a user to select or for the sleep-aid device 102 to select or recommend for the user based on the user's sleep disorder, for example. Other factors may be utilized by the sleep-aid device 102 to select or recommend a theme, as well.

The first audio content may be played following the audiovisual content 110 so as to continue aiding the user 104 with falling asleep. The second audio may be played throughout sleep at predicted and/or determined sleep states determined by the sleep-aid device 102 sensing biological activity. That is, the second audio may be repeated multiple times based on the predicted and/or determined sleep states (e.g., REM state). In one embodiment, the sleep-aid device 102 may be configured to sense the biological activity. The third audio content may be played in response to sensing that the user 104 is waking up.

The sleep-aid device 102 may include sensors for the sensing biological activity. The sensors may include, but are not limited to, accelerometers, actigraphs, motion sensors, image sensors, infrared sensors, ultrasound sensors, microphones, and/or other sensors configured to sense biological activity. The sensor(s) may be integrated within the sleep-aid device 102 or be communicatively connected to the device 102 wirelessly or via wire. If not integrated with the device, the sensor(s) may be physically attached or detached. If detached, then it may be possible for the device 102 to perform the sleep-aid functionality without being physically on the moveable surface 106 if the sensor(s) are placed on the moveable surface 106 or possibly attached to the user 104. One of skill in the art will appreciate that many sensors and sensing methods exist for sensing biological activity during sleep. The sensors may be configured to communicate signals indicative of the sensed biological activity to a processing unit of the sleep-aid device 102. The processing unit may be configured to cause a display and an audio output device to play either the second or third audio content based on the sensed biological activity. It should be understood that alternative configurations in which the sleep-aid device 102 is configured to not simply lay on a moveable surface 106 may be used. Such configurations may include a strap or band that supports the device to be positioned on the user 104, for example.

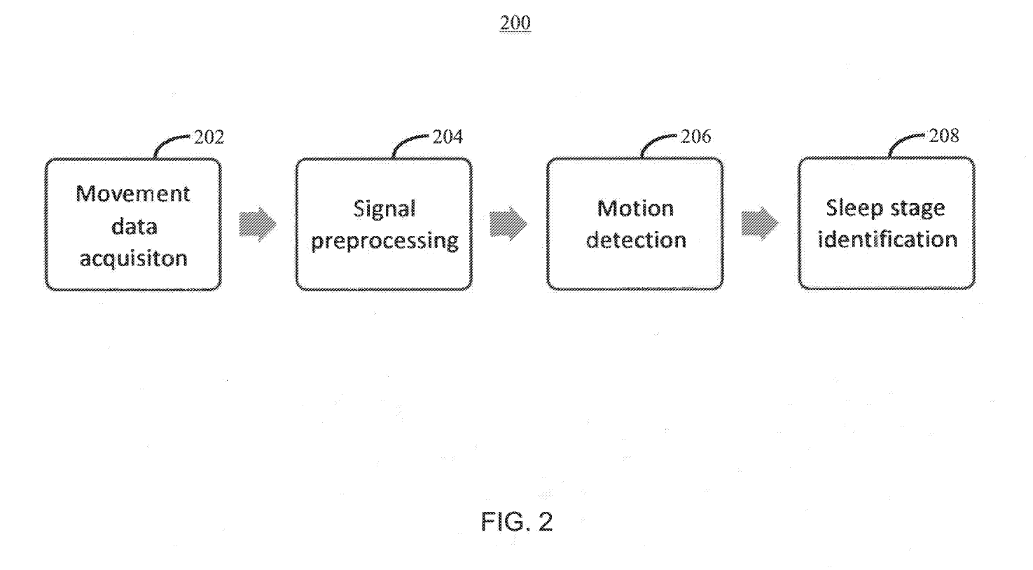

With regard to FIG. 2, a process 200 of aiding sleep based on acquisition of biological activity of a user through sensors is shown. The process 200 may be implemented by a sleep-aid device, such as the sleep-aid device 102 of FIG. 1. The sleep-aid device (or sensor(s) in communication therewith) may be placed on a moveable surface near a position on the moveable surface where the user's head rests while sleeping, for example. Alternative positions of the sleep-aid device 102 on a moveable surface may work, as well. Movement data may be acquired and/or sampled at step 202. In one embodiment, data may be sampled at a 2 Hertz frequency. In other embodiments, different sampling rates may be used. Each sample of data may include real values representative of biological activity. In one embodiment, the data may include three real values representative of acceleration in three accelerometer axes. It should be understood that alternative sensing configurations and parameters may be used to sense or more biological activities (e.g., movement, sound, etc.) of the user..

At step 204, the data may be preprocessed. If a three-axis sensing device is used, then for each axis, an average may be calculated within a moving time window, for example. In one embodiment, the moving time window may include a ten second moving time window. Data samples that may be less than four standard deviations away from the calculated average may be considered measurement noise. In one embodiment, three data samples or three processed data samples may be summed together to obtain one signal. At step 206, motion may be detected by taking the calculated signal of step 204 and applying a dynamic threshold to obtain discrete information about movement. At step 208, information about the movement may be processed by the sleep-aid device. The sleep-aid device may then determine a most probable current sleep state. A variety of statistical analysis functions may be utilized.

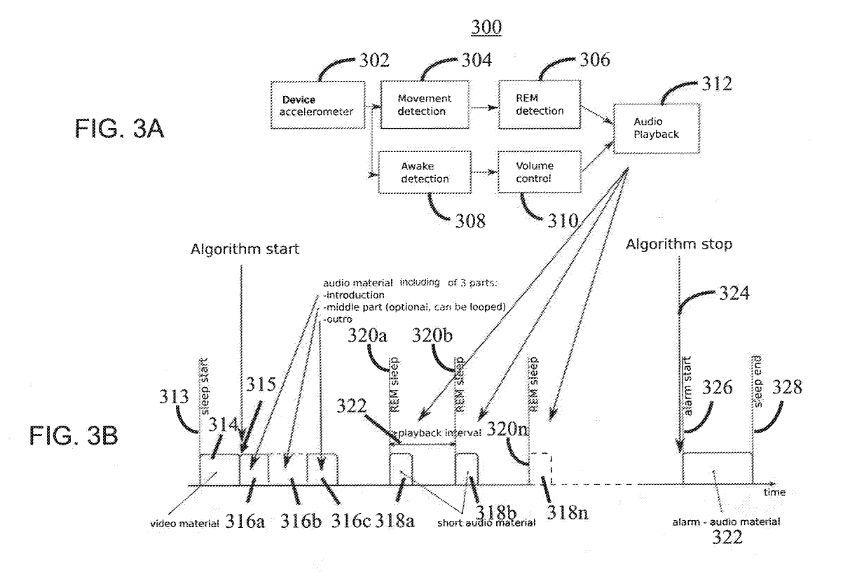

With regard to FIGS. 3A and 3B, flow diagrams of an illustrative process 300 that represents a general flow of a sleep-aid system described herein are shown. The process 300 may include playing an audiovisual and three audio contents to assist a user in at least one sleep state, including falling asleep, staying asleep, preventing nightmares, and waking up. In one embodiment, the audiovisual and three audio contents are the audiovisual and three audio contents that have a common theme of FIG. 1. Prior to the process 300 beginning, at least one device 302, such as an accelerometer, may be configured to detect movement, and movement detection may be made at step 304. In response to detected movement, a determination may be made as to whether the user is in a rapid eye movement (REM) state of sleep at step 306. The process 300 may further include detecting if the user is waking up at step 308. In response to detecting that the user is waking up, volume of audio being or to be played to a user may be controlled in a manner that increases, decreases, or remains the same at step 310. An audio content may be played at step 312 based on data from the REM determination of step 306 and the volume control of step 310.

The process 300 may begin at step 313 in response to a user initiated start that initiates playing of audiovisual content at step 314. At step 315, a sleep-aid algorithm may start a first audio content that includes playing three parts of audio at steps 316a, 316b, and 316c (collectively 316). The first part of the first audio content may be played at step 316a. In one embodiment, the first audio part may be an introduction, and may be configured to play for a predetermined amount of time, such as two minutes. In one embodiment, the user may set a length of playtime for the first audio content. A second part of the audio content may be played at step 316b. In one embodiment, the second audio part may be a middle section and may be configured to play for a predetermined amount of time, such as two-and-a-half minute repeatable loops, where the number of repeated loops may be established to be static (e.g., user sets number of minutes) or dynamic, where the sleep-aid device may determine length of time (and number of loops) based on biological measurement(s). A third part of the audio content may be played at step 316c, and played in response to sensing that the user is waking up. In one embodiment, the third audio part may be a conclusion, and may be configured to play for a predetermined amount of time, such as three minutes. The length may be matched by playing the first audio part and third audio part a single time and looping the second audio part.

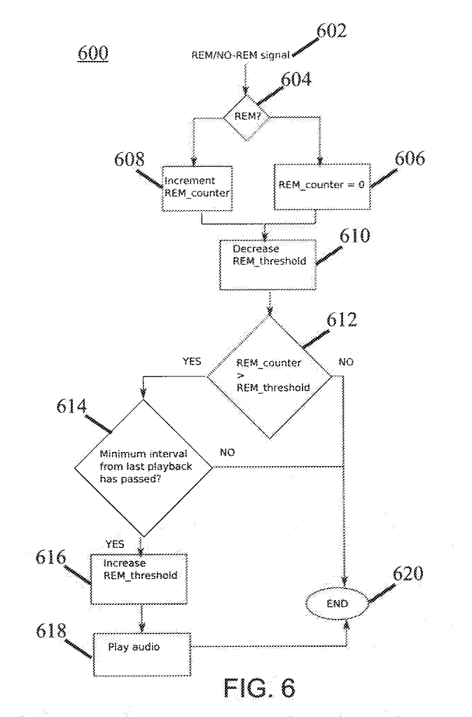

More specifically, in response detecting REM sleep at step 306 (FIG. 3A), a playback interval may begin at steps 320a, 320b, 620c (collectively 320). The playback interval 620 may play the second audio content at steps 318a, 318b, 318c (collectively 318). In one embodiment, the playback interval 320 may be adjusted according to process 600 of determining a playback value of FIG. 6.

In response to detecting that the user is waking up at step 308, the process 300 may begin a conclusion process at time 324. Time 324 may be set relatively soon (e.g., within 10 seconds) after detecting that the user is waking up. An alarm start time 326 may begin playing the third audio content at step 322 to assist the user in waking up. The process 300 may end when the user has finished sleeping at a sleep end time 328. In one embodiment, diagnostics of the process 300 may be presented to the user on a an electronic display, such as on an electronic device that the process 300 is executing. In another embodiment, the user may input data regarding sleep during the process 300 to a user interface. In one embodiment, the process 300 may be executed by a processing unit of a sleep-aid device (e.g., smartphone) in combination with sensors, electronic display, and speaker.

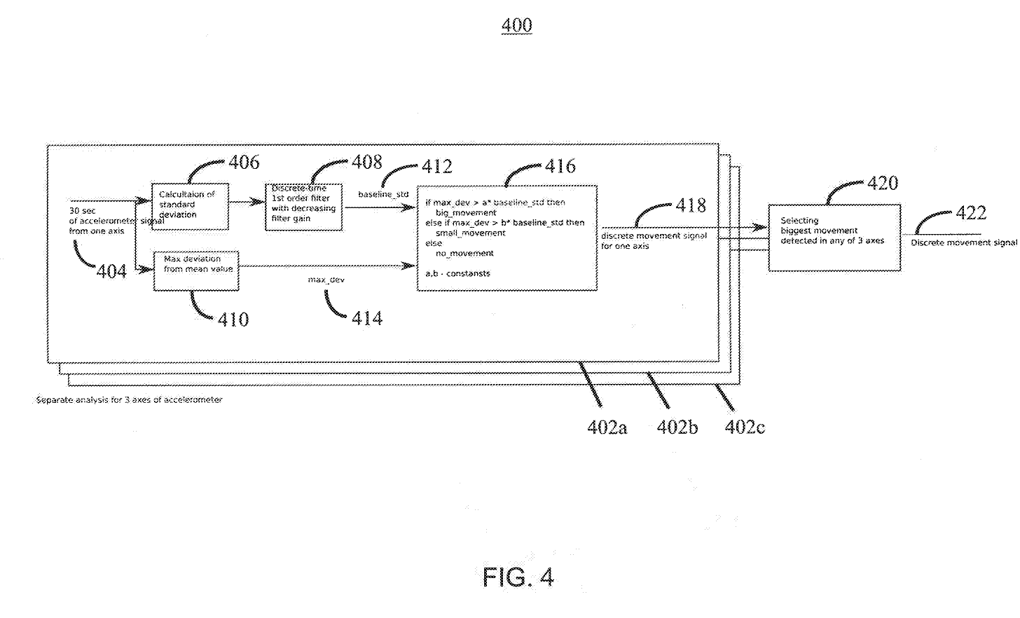

With regard to FIG. 4, a process 400 of determining biological activity based on detecting motion of a sleeper is shown. The process 400 is composed of multiple analyses 402a, 402b, and 402c (collectively 402) that are performed for sensing motion sensed by accelerometers in three axes. The analysis 402a may include receiving an accelerometer signal from a corresponding accelerometer at step 404. The accelerometer signal may be collected over a predetermined time period, such as 30 seconds. A standard deviation may be calculated at step 406. The signal may be processed by a discrete-time, first order filter with decreasing filter gain at step 408 to remove any spurious peaks that might be indicative of false movement by the user. Additionally, a maximum deviation from mean value of the signal may be calculated at step 410.

A baseline signal 412 and a maximum deviation signal 414 may be fed into a comparison algorithm at step 416. In one embodiment, in response to a value represented by the maximum deviation signal 414 being greater than a value represented by the baseline signal 412 multiplied by a first constant or scale factor, the detected motion may be categorized as a big movement. In response to the value represented by the maximum deviation signal 414 being greater than a value represented by the baseline signal 412 multiplied by a second constant, the detected motion may be categorized as a small movement. In one embodiment, in response to the value represented by the maximum deviation signal 414 being less than the value represented by the baseline signal 412 multiplied by the first constant and the baseline signal 412 multiplied by the second constant, the detected motion may be categorized as no movement. The same processing may be performed for each of analyses 402b and 402c. However, the first and second constants may be the same or different for the different axes that may sense the same or different levels of motion from a user during sleep.

A discrete movement signal 418 formed from the maximum deviation signals 414 may result from the comparison algorithm of step 416. At step 420, the discrete movement signal 418 from each of analyses 402a, 402b, and 402c may be evaluated and a biggest movement detected. A discrete movement signal 422 may be an output of the process 400. In one embodiment, the process 400 may be executed by a processing unit of a sleep-aid device.

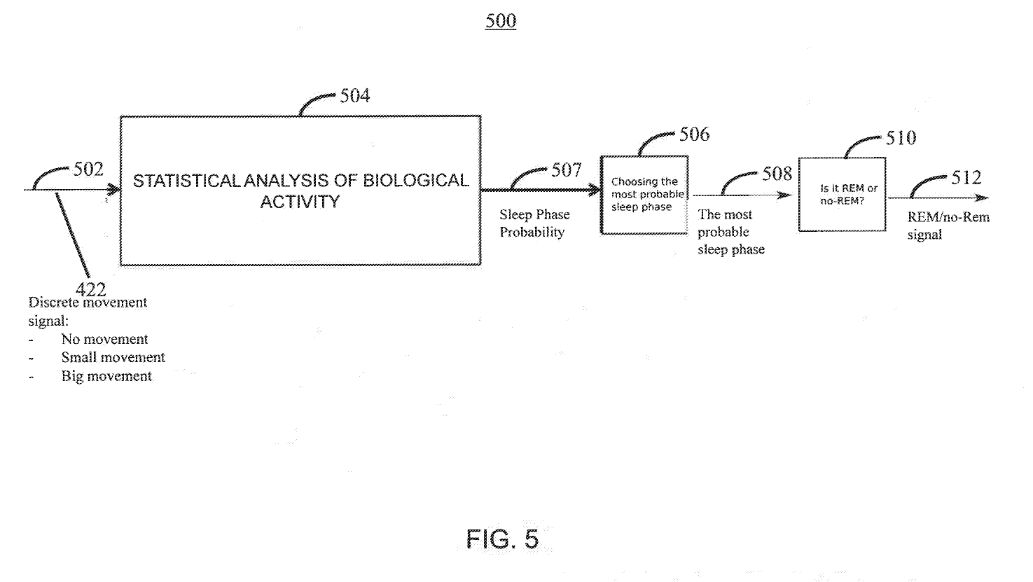

With regard to FIG. 5, a process 500 of determining a state of sleep based on statistical analysis of biological activity is shown. In an embodiment, a discrete movement signal 502, which may be the same or similar to the discrete movement signal 422 of FIG. 4, may be received. The discrete movement signal 522 may be representative of (i) no movement, (ii) small movement, or (iii) big movement, as previously described. Statistical analysis of the discrete movement signal 502 may be performed at step 504 to determine a sleep phase probability 507. The statistical analysis may use a variety of mathematical probability functions to enable statistically determining a state or phase of sleep (i.e., sleep phase probability 507)of the user. At step 506, a most probable sleep state 508 may be chosen based on the sleep phase probability 507 (e.g., one or more data values) of the discrete movement signal 422. At step 510, a determination may be made of whether the most probable sleep state 508 is REM or non-REM, and a sleep state signal 512 may be stored, transmitted, or otherwise used. In one embodiment, the sleep state signal 512 is representative of one of a REM state and a non-REM state.

With regard to FIG. 6, a process 600 of determining a playback counter value based on a predicted state of sleep is shown. The process 600 may begin at step 602, where a rapid eye movement (REM)/non-REM signal is received. At step 604, a determination of the user being in one of a REM state and a non-REM state of sleep may be made based on the REM/non-REM signal. In response to the determination being that the user is in a non-REM state, a REM counter may be set to zero at step 606. In response to the determination being that the user is in a REM state, the REM counter may be incremented at step 608. A REM threshold may be decreased at step 610.

At step 612, a determination may be made if the REM counter is greater than the REM threshold. In response to determining that the REM counter is greater than the REM threshold, a determination of whether a minimum interval from a previous playback has passed may be made at step 614. In response to determining that the REM counter is less than the REM threshold at step 612, the process 600 may end at step 620.

In response to a determination that the minimum interval from the previous playback has passed at step 614, the REM threshold may be increased at step 616. An audio content, such as the second audio content of FIG. 1, may be played at step 618, and the process 600 may end at step 620. In response to a determination at step 614 that the minimum interval from the previous playback has not passed, the process 600 may end at step 620.

The ability to determine whether a user is in a REM state has been found to be difficult by researchers even with highly sophisticated sleep monitoring equipment. In an embodiment, the principles described herein provide for monitoring certain movements and using statistical analyses to help determine whether those sensed movements are indicative of a user dreaming or not dreaming. Based on determining whether or not the movements are associated with dreams, a statistical analysis may be performed as to whether or not the user is in a REM state. It has been found that, using the principles of the sleep-aid device described herein that such an analysis technique has a surprisingly high success rate of determining whether a user is in a REM state as the use of much more sophisticated sleep analysis equipment has about the same or less effectiveness in identifying when a user is in a REM state, as further described with regard to FIG. 5.

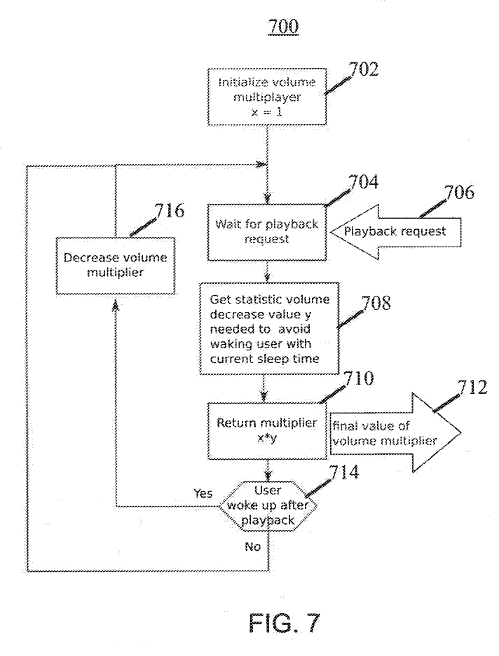

With regard to FIG. 7, a flow diagram of an illustrative a process 700 of determining a volume to play an audio content in response to biological response of a user to the audio content is shown. The process 700 may be executed by a processor of an electronic device, as previously described. The volume control process 700 may be the volume control of step 310 of FIG. 3. The process 700 may initialize a volume multiplier by setting the volume multiplier to one at step 702. At step 704, the process 700 may wait for a playback request 706. At step 708, a statistical volume decrease value used to avoid waking the user with current sleep time may be accessed. The volume multiplier and volume decrease value may be multiplied together at step 710 to determine a new volume multiplier 712. A signal representative of the determined volume multiplier 712 may be transmitted to a processing unit (e.g., processor of a mobile electronic device) controlling audio output.

A determination may be made if the user wakes up after playback at step 714. In response to the user waking up, the volume multiplier may be decreased at step 716, and the process 700 may return to waiting for a playback request at step 704. In response to the user not waking up, the process 700 may return to waiting for the playback request at step 704.

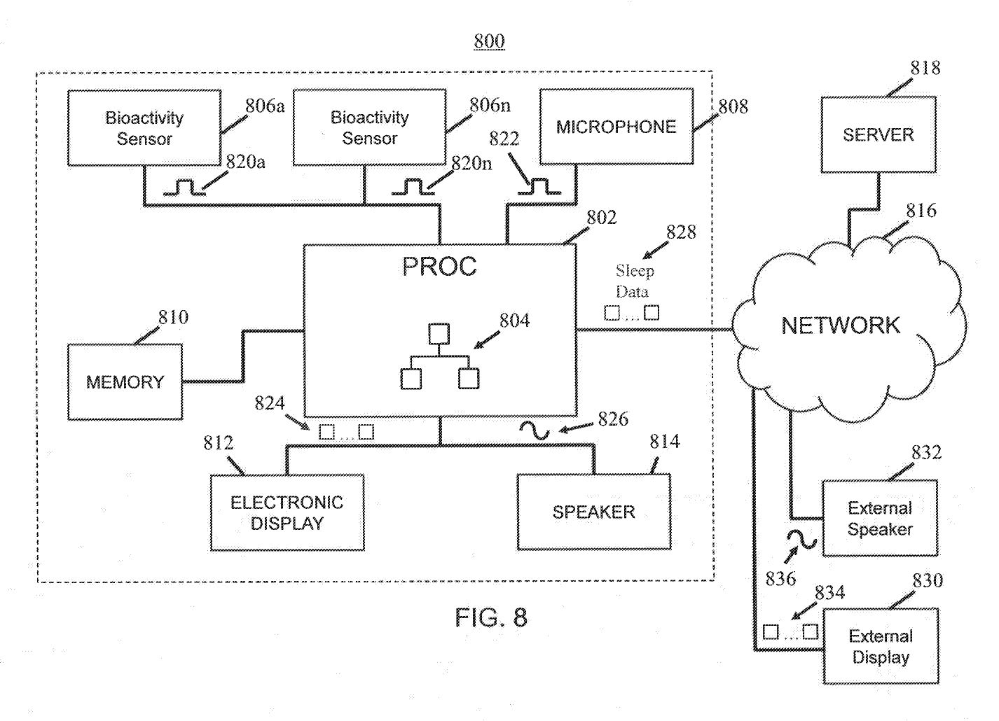

With regard to FIG. 8, electronics of a sleep-aid system 800 configured to aid a user while sleeping are shown. The sleep-aid system 800 may include a processing unit 802 configured to execute software 804. The processing unit 802 may be a computer proc3essor, such as a general processor, digital signal processor, application specific integrated circuit (ASIC), or any other electronic circuit configured to execute computer instructions. The processing unit 802 may be in electrical communication with bioactivity sensors 806a-806n (collectively 806) configured to sense biological activity, a microphone 808 for sensing sound, a memory unit 810 for storing data, an electronic display 812, and a speaker 814. In one embodiment, the sleep-aid system 800 may communicate via a network 816 with a remote server 818.

The bioactivity sensors 806 may be configured to store and/or communicate biological activity signals 820a-820n (collectively 820) representative of data indicating biological activity sensed by the bioactivity sensors 806 to the processing unit 802. The microphone 808 may communicate a sound signal 822 representative of data indicating a sensed biological sound to the processing unit 802. The processing unit 802 may be configured to communicate an audiovisual data packet 824 to the electronic display 812 to be played for the user to watch to aid the user in falling asleep. The processing unit 802 may be further configured to communicate a sound wave 826 representative of audio content to be played so as to aid the user in at least one of falling asleep, staying asleep, waking up, and reducing or preventing nightmares and/or other sleep disorders, to the speaker 814. Sleep data 828 may be communicated to the server 818 via the network 816 to create and maintain user profiles for users. In one embodiment, the sleep data 828 may assist updating sleep-aid processes used by the processing unit 802.

In one embodiment, the electronic display 812 and the speaker 814 are internal electronic components of a sleep-aid device housing the processing unit 802. In another embodiment, an additional external display 830 and an additional external speaker 832 may be in wireless electrical communication with the processing unit 802. The processing unit 802 may be configured to communicate the audiovisual content 834 and the audio content 836 to the external display 830 and the external speaker 832 via the network 816. In yet another embodiment, a sleep-aid device housing the processing unit 802 may not include the electronic display 812 and the speaker 814. The processing unit 802 may be configured to communicate only with the external display 830 and the external speaker 832. One of skill in the art will appreciate that many configurations of internal and external sensors and output devices may achieve a substantially similar sleep-aid system.

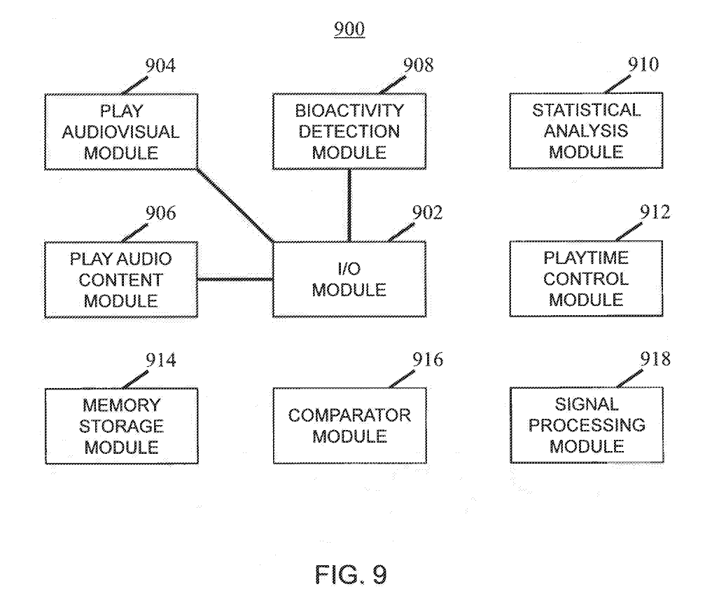

With regard to FIG. 9, a block diagram inclusive of illustrative software modules 900 that may be executed by a processing unit of a sleep-aid system, such as according to FIG. 8, that is configured to aid a user in sleeping is shown. The software modules 900 may include an input/output (I/O) module 902 for managing inputs and outputs of the sleep-aid system. In one embodiment, the inputs may include, but are not limited to, sensor signals, signals from sensors and communication and/or interaction from the user via a user interface or otherwise. The processing unit may store data in a memory unit of the sleep-aid system representative of the signals from the inputs. In one embodiment, the outputs may include, but are not limited to, sensor signals, signals from the processing unit to at least one of an electronic display and an audio output device, audio and audiovisual signals to a local device (e.g., tablet or television), remote server, etc.

The I/O module 902 may be configured to send data to a play audiovisual module 904 and play audio content module 906. The play audiovisual module 904 may be configured to play audiovisual content, such as the audiovisual content described herein, on an electronic display. The play audio content module 906 may be configured to play a first audio content, a second audio content, and a third content, as previously described. A bioactivity module 908 may be configured to process sensor signals received from a bioactivity sensor and communicate a signal to the processing unit corresponding to the sensed biological activity.

A statistical analysis module 910 may include receiving data and performing various statistical analyses on the data. The statistical analysis module 910 may include determined statistical analyses by a user and/or a set of statistical calculations performed on each set of data received by the statistical analysis module 910. A playtime control module 912 may control a length of overall playtime of the audio content. The playtime control module 912 may receive a determined amount of time from a user representative of a preferred playtime of the user. The playtime module 912 may calculate a number of times to loop a middle portion of the audio content to achieve the playtime requested by the user or determined by the sleep-aid device.

A memory storage module 914 may be configured to store audiovisual content and audio content, where the content may have metadata inclusive of themes to enable selection of commonly themed content. The memory storage module 914 may also store software and data collected and generated in performing the sleep-aid processes. A comparator module 916 may be configured to compare data points and to return a signal representative of the comparison (e.g., determining if value A is greater than value B). The comparator module 916 may further be configured to return a number of signal outputs, such as, but not limited to, the data point that is either greater or less than a Boolean value indicating that the comparison was true or false, and others understood by those of skill in the art. A signal processing module 918 may process signals representative of biological activity that may be received from the bioactivity detection module 908. One of skill in the art will appreciate that additional and/or alternative modules may exist for the processes described herein.

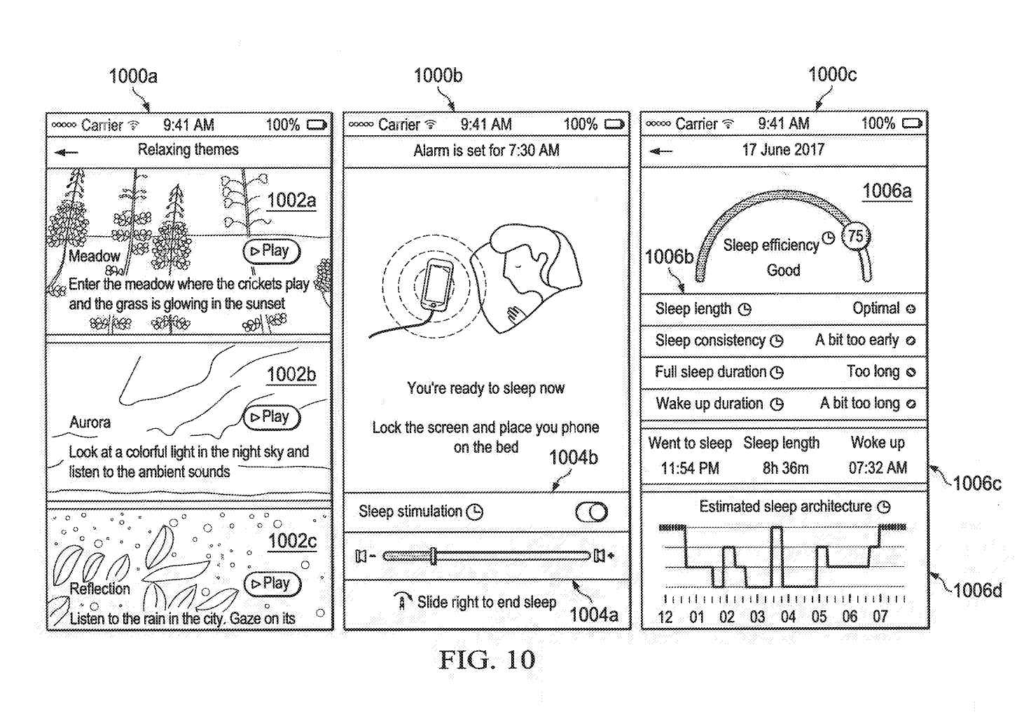

With regard to FIG. 10, illustrative screenshots 1000a, 1000b, 1000c (collectively 1000) of an application on a sleep-aid device configured to interface with a user regarding a sleep-aid application of the sleep-aid device is shown. Screenshot 1000a may include a theme selection page. Screenshot 1000b may include a user control settings page. Screenshot 1000c may include a sleep analysis page.

Screenshot 1000a may include themes 1002a, 1002b, 1002c (collectively 1002) that may be available for a user to select. In one embodiment, a theme 1002 may include a combination of an audiovisual content, a first audio content, a second audio content, and a third audio content. In one embodiment, themes 1002 may be developed prior to user access and may be configured to optimize relaxation and assisting users while falling asleep.

Screenshot 1000b may include settings 1004a, 1004b (collectively 1004) that may affect a user's experience. In one embodiment, the settings 1004 may include a volume control setting 1004a that may be configured to control a volume that audio content of the theme 1002 is played. In one embodiment, the settings 1004 may include a sleep stimulation setting 1004b to aid in staying asleep, preventing nightmares, and waking up. In one embodiment, the user control settings page may enable the user to control the settings 1004. In one embodiment, settings 1004 may include preset values based on theme 1002 selected by the user. One of ordinary skill in the art will appreciate that various additional and/or alternative settings exist that a user may control.

Screenshot 1000c may include diagnostic data 1006a, 1006b, 1006c, 1006d (collectively 1006) regarding a sleep session of the user. The diagnostic data 1006 may include, but is not limited to, a sleep efficiency rating 1006a, sleep state duration analysis 1006b, milestones 1006c that may be a prediction of a time that various sleep events occurred, and a sleep graph 1006c that may visualize the sleep session of the user. One of skill in the art will appreciate that additional diagnostic data may be displayed for and/or communicated to the user. In one embodiment, the user may determine which diagnostic data to display on the sleep analysis page. In one embodiment, the diagnostic data to be displayed on the sleep analysis page may be present based on a sleep disorder of the user. One of skill in the art will also appreciate that various additional screens may be used by a sleep-aid application. It should be understood that the data may be communicated to a remote computer.

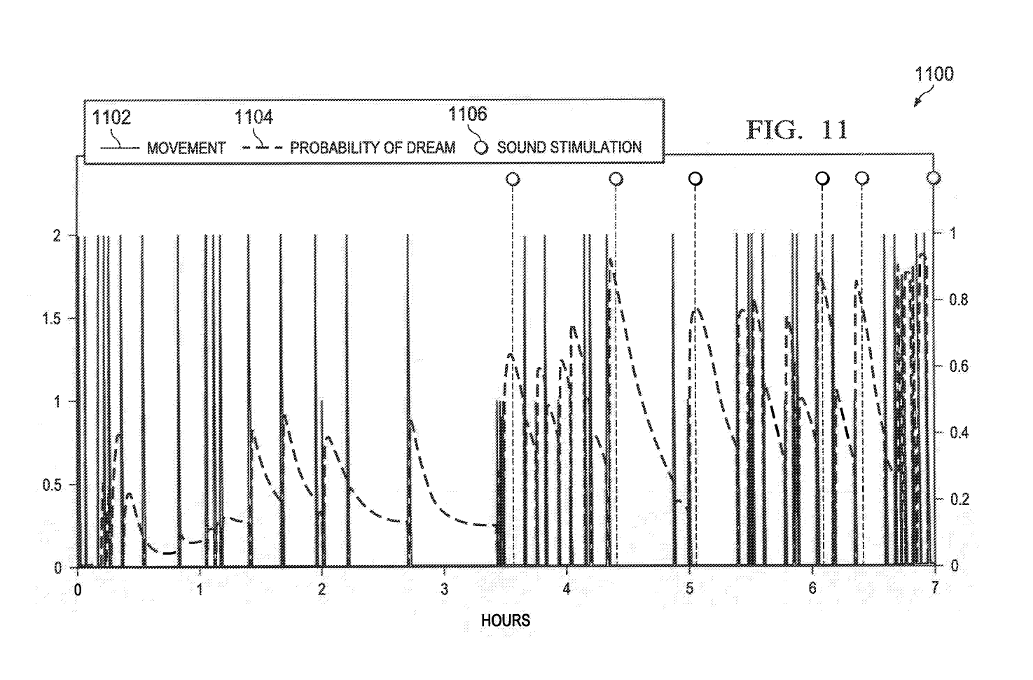

With regard to FIG. 11, a graph representative of a sleep session 1100 of a user inclusive of detected movement 1102, predicted dreams 1104, and sound stimulations 1106 is shown. In one embodiment, the predicted dreams 1104 (shown as vertical lines) may correspond with detected movement 1102. From the predicted dreams 1104, a further determination may be made as to whether or not a user is in a REM sleep, and if so, sound stimulations 1106 may be played. The sound stimulations 1106 may be a second audio content, as previously described, for a certain amount of time.

One embodiment of a computer-implemented method of aiding sleep may include selecting a common theme of audiovisual content and audio content and enabling a user to play the audiovisual content. After the audiovisual content has completed, the method may include initiating playing the first audio content by an audio output device a determined number of times to ease falling asleep. In response to receiving a first sensor signal from one of at least one sensor that senses a first biological activity indicative of a specific state during sleep, the method may include causing the second audio content to be played by the audio output device. In response to receiving a second sensor signal from one of the sensor(s) that senses a second biological activity indicative of the user beginning to wake up, the method may include causing the third audio content to be played at a first volume threshold to wake the user by the audio output device. The common theme of audiovisual and audio content may include a first audio content, a second audio content, and a third audio content.

In an embodiment, initiating playing the first audio content may include initiating playing (i) an introduction portion of the first audio content, (ii) a middle portion of the first audio content, and (iii) an outro portion of the first audio content, and further causing the middle portion to play a determined number of times to vary overall play time of the first audio content. The process may further enable the user to select the overall play time of the first audio content, and compute a number of loops to play the middle portion of the first audio content based on the overall play time selected by the user. Selecting a common theme of audiovisual content and audio content may include selecting at least one of (i) distinct portions of an audio track and (ii) separate versions of an audio track. The process may further repeat detecting whether a user is beginning to wake up from sleeping.

In response to receiving the second sensor signal from one of the sensor(s) that senses the second biological activity indicative of the user beginning to wake up, the second audio content may be caused to be played at a second volume threshold to prevent fully waking the user. The process may further be configured to select the common theme from multiple themes of audiovisual content and audio content based on metadata derived from data collected from the user. The process may further include receiving movement signals by one of the sensor(s) in any of three axes, determining a largest movement signal amongst the movement signals, and determining a biological activity from the determined largest movement signal.

The process may further include sensing a dream based on sensing motion based on signals from the sensor(s), and correlating a rapid eye movement (REM) state with the sensed dream. Moreover, the process may further include storing a REM sleep counter in a memory unit, incrementing the REM sleep counter in response to correlating the REM state with the sensed dream, and resetting the REM sleep counter to zero in response to the at least one sensor sensing the user is not in a REM state of sleep. In an embodiment, the process may further include storing a REM sleep threshold value in the memory unit, comparing the REM sleep threshold value and the REM sleep counter, and in response to the REM sleep counter (i) being greater than the REM sleep threshold value and (ii) passing a minimum interval from a previous playback of the second audio content, increasing the REM sleep threshold value.

The process may further include applying a statistical analysis to a signal from the sensor(s) to determine a state during sleep. Still yet, the process may include storing a volume multiplier representative of a volume of the second audio content that will not fully wake up the user when the user begins to wake up in a memory unit, and in response to user fully waking up during playing of the second audio content, decreasing the volume multiplier.

The foregoing method descriptions and the process flow diagrams are provided merely as illustrative examples and are not intended to require or imply that the steps of the various embodiments must be performed in the order presented. As will be appreciated by one of skill in the art, the steps in the foregoing embodiments may be performed in any order. Words such as “then,” “next,” etc. are not intended to limit the order of the steps; these words are simply used to guide the reader through the description of the methods. Although process flow diagrams may describe the operations as a sequential process, many of the operations may be performed in parallel or concurrently. In addition, the order of the operations may be re-arranged. A process may correspond to a method, a function, a procedure, a subroutine, a subprogram, etc. When a process corresponds to a function, its termination may correspond to a return of the function to the calling function or the main function.

The various illustrative logical blocks, modules, circuits, and algorithm steps described in connection with the embodiments disclosed here may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to and/or in communication with another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

The actual software code or specialized control hardware used to implement these systems and methods is not limiting of the invention. Thus, the operation and behavior of the systems and methods were described without reference to the specific software code being understood that software and control hardware can be designed to implement the systems and methods based on the description here.

When implemented in software, the functions may be stored as one or more instructions or code on a non-transitory computer-readable or processor-readable storage medium. The steps of a method or algorithm disclosed here may be embodied in a processor-executable software module which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used here, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The previous description is of a preferred embodiment for implementing the invention, and the scope of the invention should not necessarily be limited by this description. The scope of the present invention is instead defined by the following claims.

1. A sleep aid device, comprising:
at least one sensor configured to sense an activity of a user during sleep;
a memory unit configured to store audiovisual and audio content available to be (i) selected for display on an electronic display and (ii) selected for play by an audio output device; and
a processing unit in communication with said at least one sensor and said memory unit, and configured to:
select a common theme of audiovisual content and audio content including:
(i) a first audio content;
(ii) a second audio content; and
(iii) a third audio content;
enable the user to play the audiovisual content on the electronic display;
after the audiovisual content has completed, initiate playing the first audio content by the audio output device a determined number of times to ease falling asleep;
in response to receiving a first sensor signal from one of said at least one sensor that senses a first biological activity indicative of a specific state during sleep, cause the second audio content to be played by the audio output device; and
in response to receiving a second sensor signal from one of said at least one sensor that senses a second biological activity indicative of the user beginning to waking up, cause the third audio content to be played at a first volume threshold to wake the user by the audio output device.

2. The sleep aid device according to claim 1, wherein the first audio content includes an introduction portion, a middle portion, and an outro portion, said processing unit configured to cause the middle portion to play a determined number of times to vary overall play time of the first audio content.

3. The sleep aid device according to claim 2, wherein said processing unit is further configured to:
enable the user to select the overall play time of the first audio content; and
compute a number of loops to play the middle portion of the first audio content based on the overall play time selected by the user.

4. The sleep aid device according to claim 1, wherein the first, second, and third audio contents are at least one of (i) distinct portions of an audio track and (ii) separate versions of an audio track.

5. The sleep aid device according to claim 1, wherein said processing unit is further configured to repeat detecting whether a user is beginning to wake up from sleeping.

6. The sleep aid device according to claim 1, wherein said at least one sensor includes at least one accelerometer, said processing unit being further configured to determine biological activity in response to sensing motion based on signals from the accelerometer.

7. The sleep aid device according to claim 1, wherein said processing unit is further configured to, in response to receiving the second sensor signal from one of said at least one sensor that senses the second biological activity indicative of the user beginning to wake up, cause the second audio content to be played at a second volume threshold to prevent fully waking the user.

8. The sleep aid device according to claim 1, wherein said processing unit is further configured to select the common theme from a plurality of themes of audiovisual content and audio content based on metadata derived from data collected from the user and stored in said memory unit.

9. The sleep aid device according to claim 1, wherein said processing unit is further configured to:
receive movement signals by one of said at least one sensor in any of three axes;
determine a largest movement signal amongst the movement signals; and
determine a biological activity from the determined largest movement signal.

10. The sleep aid device according to claim 1, wherein said processing unit is further configured to:
correlate a rapid eye movement (REM) state with motion signal from one of said at least one sensor.

11. The sleep aid device according to claim 10, wherein said processing unit is further configured to:
store a REM sleep counter in said memory unit;
increment the REM sleep counter in response to correlating the REM state with the sensed dream; and
reset the REM sleep counter to zero in response to said at least one sensor sensing the user is not in a REM state of sleep.

12. The sleep aid device according to claim 11, wherein said processing unit is further configured to:
store a REM sleep threshold value in said memory unit;
compare the REM sleep threshold value and the REM sleep counter; and
in response to the REM sleep counter (i) being greater than the REM sleep threshold value and (ii) passing a minimum interval from a previous playback of the second audio content, increase the REM sleep threshold value.

13. (canceled)

14. The sleep aid device according to claim 1, wherein said processing unit is further configured to:
store a volume multiplier representative of a volume that will not fully wake up the user when the user begins to wake up in said memory unit; and
in response to user fully waking up during playing of the second audio content, decrease the volume multiplier.

15. (canceled)

16. (canceled)

17. A computer-implemented method of aiding sleep, the method comprising:
selecting a common theme of audiovisual content and audio content including:
(i) a first audio content;
(ii) a second audio content; and
(iii) a third audio content;
enabling a user to play the audiovisual content;
after the audiovisual content has completed, initiating playing the first audio content by an audio output device a determined number of times to ease falling asleep;
in response to receiving a first sensor signal from one of at least one sensor that senses a first biological activity indicative of a specific state during sleep, causing the second audio content to be played by the audio output device; and
in response to receiving a second sensor signal from one of the at least one sensor that senses a second biological activity indicative of the user beginning to waking up, causing the third audio content to be played at a first volume threshold to wake the user by the audio output device.

18. The computer-implemented method according to claim 17, wherein the initiating playing the first audio content includes initiating playing an introduction portion of the first audio content, a middle portion of the first audio content, and an outro portion of the first audio content, and further causing the middle portion to play a determined number of times to vary overall play time of the first audio content.

19. The computer-implemented method according to claim 18, further comprising:
enabling the user to select the overall play time of the first audio content; and
computing a number of loops to play the middle portion of the first audio content based on the overall play time selected by the user.

20. The computer-implemented method according to claim 17, wherein said selecting a common them of audiovisual content and audio content includes selecting at least one of (i) distinct portions of an audio track and (ii) separate versions of an audio track.

21. The computer-implemented method according to claim 17, further comprising repeating detecting whether a user is beginning to wake up from sleeping,

22. The computer-implemented method according to claim 17, further comprising, in response to receiving the second sensor signal from one of the at least one sensor that senses the second biological activity indicative of the user beginning to wake up, causing the second audio content to be played at a second volume threshold to prevent fully waking the user.

23. The computer-implemented method according to claim 17, further comprising selecting the common theme from a plurality of themes of audiovisual content and audio content based on metadata derived from data collected from the user and stored in a memory unit.

24. The computer-implemented method according to claim 17, further comprising:
receiving movement signals by one of the at least one sensor in any of three axes;
determining a largest movement signal amongst the movement signals; and
determining a biological activity from the determined largest movement signal.

25. The computer-implemented method according to claim 17, further comprising:
sensing a dream based on sensing motion based on signals from the at least one sensor; and
correlating a rapid eye movement (REM) state with the sensed dream.

26. The computer-implemented method according to claim 25, further comprising:
storing a REM sleep counter in a memory unit;
incrementing the REM sleep counter in response to correlating the REM state with the sensed dream; and
resetting the REM sleep counter to zero in response to the at least one sensor sensing the user is not in a REM state of sleep.

27. The computer-implemented method according to claim 26, further comprising:
storing a REM sleep threshold value in the memory unit;
comparing the REM sleep threshold value and the REM sleep counter; and
in response to the REM sleep counter (i) being greater than the REM sleep threshold value and (ii) passing a minimum interval from a previous playback of the second audio content, increasing the REM sleep threshold value.

28. (canceled)

29. The computer-implemented method according to claim 17, further comprising:
storing a volume multiplier representative of a volume of the second audio content that will not fully wake up the user when the user begins to wake up in a memory unit; and
in response to user fully waking up during playing of the second audio content, decreasing the volume multiplier.
